(19) United States
(12) Patent Application Publication
(10) Pub. No.: US 2024/0325347 A1
(43) Pub. Date: Oct. 3, 2024

(54) ZINC ASCORBATE, HESPERIDIN, ARGININE, QUERCETIN AND CALCIDIOL CONTAINING COMPOSITIONS FOR SUPPORTING IMMUNE HEALTH

(71) Applicant: SystaMedic Inc., Clinton, CT (US)

(72) Inventor: Anton Franz Joseph Fliri, Mystic, CT (US)

(73) Assignee: SystaMedic Inc., Clinton, CT (US)

(21) Appl. No.: 18/616,523

(22) Filed: Mar. 26, 2024

Related U.S. Application Data
(60) Provisional application No. 63/502,453, filed on May 16, 2023.
(60) Provisional application No. 63/493,469, filed on Mar. 31, 2023.

(51) Int. Cl. A61K 31/353; A61K 31/047; A61K 31/198; A61K 31/7048; A61K 33/30; A61K 45/06

(52) U.S. Cl. CPC A61K 31/353; A61K 31/047; A61K 31/198; A61K 31/7048; A61K 33/30; A61K 45/06

(57) ABSTRACT

The present disclosure relates to compositions as well as methods of use and methods of treating, including zinc ascorbate, hesperidin, quercetin, arginine, and calcidiol.

### FIELD

The aspects of the present disclosure relate to compositions and methods including zinc ascorbate, hesperidin, quercetin, arginine, and calcidiol.

### BACKGROUND

Strengthening the immune system without causing negative effects resulting from immune system hyperactivation or hypoactivation requires a careful balance between different immune cells. Functions of immune cells involved are regulated in part by the ratio between Th1/Th2 and Th17/Treg cells wherein regulatory T cells (Tregs) are vital in maintaining healthy balances between these immune cells [Journal of neuroimmunology 337 (2019): 577071]. Affecting regulatory functions mediated by T cells are oxidative stress, cytokines such as Tumor Necrosis Factor alpha (TNF alpha) [Mehta A K, et al. Cytokine. 2018;101:14-18]. Tumor derived growth factors beta1 and beta 2 [Wan YY, et al. Immunol Rev. 2007 December;220:199-213] and interleukins such as interleukin 10 [Trinchieri G. J Exp Med. 2007; 204 (2): 239-243.] It has been shown that Zinc mediated signaling is important for Treg function and is crucial for T cell differentiation [Maywald M et al. Int J Mol Sci. 2018 Nov. 13; 19 (11): 3575] and that Zinc ascorbate affects T cell functions by modulating oxidative stress responses [Clin Cosmet Investig, Dermatol. 2012; 5:135-40]. Likewise, it has been shown that the amino acid arginine, supports nitric oxide production, and increases Th1/Th2 ratio, and reduces Th17/Treg ratio. [Yeh, Chiu-Li, et al. Nutrients 12.4 (2020): 1047]. Furthermore, It has been shown that Vitamin D, also known as the prohormone calcidiol or Vitamin D3 which is the biologically active hormone calcitriol, regulates ratios between Th17/Treg cells. [Zhao, Rui, et al. Frontiers in Immunology 12 (2021): 352]. Furthermore, it has been shown that the flavonoid quercetin supports maintenance of beneficial balances between Th1/Th2 and Treg/Th17cells [Ke X, et al. Autoimmunity. 2023; 56 (1): 2189133.], that the flavonoid hesperidin regulates Th1/Th2 balances [Kim S H et al. Mediators Inflamm. 2011; 2011:485402.] and down regulates inducible nitric oxide productions and oxidative stress responses [Xiaoting L, et al. Adv Exp Med Biol. 2010; 664:193-201]. In addition, it has been shown that 5-hydroxymethyl-2-furfural also known as Hydroxymethylfurfural (HMF), or 5-(hydroxymethyl) furfural, an organic compound formed by the dehydration of reducing sugars, attenuates inflammatory responses by regulating the Th1/Th2 balance [Guan D, Li Y, Cui Y, et al. Acta Biochim Biophys Sin (Shanghai). 2023; 55 (8): 1222-1233.] and that HMF positively affects ion and water homeostasis of red blood cells [Hannemann Aet al. J Physiol. 2014; 592 (18): 4039-4049]. Likewise it has been shown that punicalagin, a natural products isolated from grapefruit peels has broad anti-inflammatory effects by increasing IL10 levels [Liana de O. Trovão, et al., Journal of Immunology Research, vol. 2023, Article ID 2868707.] We have also recently shown that arginine in combination with vitamin D activates caveola mediated endocytosis [Fliri AF, Kajiji S. Front Nutr. 2022; 9:885364] and that the activation of caveola mediated endocytosis supports a reciprocal cellular feedback loops that regulates oxidative stress responses, transforms the prohormone Vitamin D calcidiol into the active form calcitriol, and lowers cellular levels of Tumor derived growth factor beta 2 (TGFB2) and thereby decreases immunosuppressive properties of TGFB2. Therefore, compositions containing combinations of L arginine, calcidiol, the mineral zinc, flavonoids quercetin and hesperidin, the antioxidants vitamin C, hydroxymethylfurfural and punicalagin by affecting Th1/Th2 and Treg/Th17cells balances, oxidative stress responses, TGFB activities, TNF alpha levels, interleukin 10 levels and properties of red blood cells have utility in supporting immune system functions and to ameliorate conditions associated with immune system imbalances in acute and chronic diseases and aging [Weyand C M, et al. Ann Am Thorac Soc. 2016; 13 Suppl 5 (Suppl 5): S422-S428. doi: 10.1513/AnnalsATS.201602-095AW].

### SUMMARY

In one embodiment, a composition is provided. The composition includes hesperidin, quercetin, L-arginine, calcidiol, and zinc ascorbate wherein the amount of L-arginine in said compositions is from about 900 mg to about 1000 mg, the amount of hesperidin is from about 100 mg to about 500 mg, the amount of quercetin is from about 200 mg to about 800 mg, the amount of calcidiol is from about 10 mcg to about 25 mcg and the amount of zinc ascorbate is from about 30 mg to about 190 mg, wherein the total amounts of ingredients/serving (dosage) is the sum of the amounts of ingredients in a composition.

In another embodiment, a composition is provided. The composition includes hesperetin, quercetin, L-arginine, calcidiol, and zinc ascorbate wherein the amount of L-arginine in said compositions is from about 900 mg to about 1000 mg, the amount of hesperetin is from about 100 mg to about 500 mg, the amount of quercetin is from about 500 mg to about 800 mg, the amount of calcidiol is from about 10 mcg to about 25 mcg and the amount of zinc ascorbate is from about 30 mg to about 190 mg, wherein the total amount of ingredients/serving (dosage) is the sum of the amounts of individual ingredients in a composition.

In another embodiment, a composition is provided. The composition includes hesperetin, quercetin, L-arginine, calcidiol, and zinc ascorbate wherein the amount f L-arginine in said compositions is from about 900 mg to about 1000 mg, the amount of hesperetin is from about 100 mg to about 500 mg, the amount of quercetin is from about 200 mg to about 800 mg, the amount of calcidiol is from about 10 mcg to about 25 mcg and the amount of zinc ascorbate is from about 30 mg to about 190 mg, wherein the total amount of ingredients/serving (dosage) is the sum of the amounts of individual ingredients in a composition.

In another embodiment a composition is provided. The composition includes hesperidin, quercetin, L-arginine, calcidiol, 5-hydroxymethyl-2-furfural and zinc ascorbate wherein the amount of L-arginine in said compositions is from about 900 mg to about 1000 mg, the amount of hesperidin is from about 100 mg to about 500 mg, the amount of quercetin is from about 200 to about 800 mg, the amount of calcidiol is from about 10 mcg to about 25 mcg, the amount of zinc ascorbate is from about 30 mg to about 190 mg, the amount of 5-hydroxymethyl-2-furfural is from about 50 mg to about 8000 mg and one additional ingredient selected from the group consisting of myoinositol, ferulic acid, icariin, myricetin, pterostilbene, resveratrol, capsicum chinense, curcumin, punicalagins, coenzyme Q10, krill oil and herring roe oil.

In another embodiment, a composition is provided. The composition includes hesperidin, quercetin, L-arginine, calcidiol, zinc ascorbate and vitamin K wherein the amount of L-arginine in said compositions is from about 900 mg to about 1000 mg, the amount of hesperidin is from about 100 mg to about 500 mg, the amount of quercetin is from about 200 mg to about 800 mg, the amount of calcidiol is from about 10 mcg to about 25 mcg, the amount of zinc ascorbate is from about 30 mg to about 190 mg and the amount of vitamin K is about 100 mcg, wherein the total amount of ingredients/serving (dosage) is the sum of the amounts of individual ingredients in a composition.

In another embodiment, a composition is provided. The composition includes hesperidin, quercetin, L-arginine, calcidiol, zinc ascorbate and icariin wherein the amount of L-arginine in said compositions is from about 900 mg to about 1000 mg, amount of hesperidin is from about 100 mg to about 500 mg, the amount of quercetin is from about 200 mg to about 800 mg, the amount of calcidiol is from about 10 mcg to about 25 mcg, the amount of zinc ascorbate is from about 30 mg to about 190 mg and the amount of icariin is from about 10 mg to about 100 mg, wherein the total amount of ingredients/serving (dosage) is the sum of the amounts of individual ingredients in a composition.

In another embodiment, a composition is provided. The composition includes hesperidin, quercetin, L-arginine, calcidiol, zinc ascorbate and myricetin wherein the amount of L-arginine in said compositions is from about 900 mg to about 1000 mg, the amount of hesperidin is from about 100 mg to about 500 mg, the amount of quercetin is from about 200 mg to about 800 mg, the amount of calcidiol is from about 10 mcg to about 25 mcg, the amount of zinc ascorbate is from about 30 mg to about 190 mg, the amount of myricetin is from about 100 mg to about 500 mg wherein the total amount of ingredients/serving (dosage) is the sum of the amounts of individual ingredients in a composition.

In another embodiment, a composition is provided. The composition includes hesperetin, quercetin, L-arginine, calcidiol, zinc ascorbate, myricetin and myoinositol wherein the amount of L-arginine in said compositions is from about 900 mg to about 1000 mg, the amount of hesperetin is from about 100 mg to about 500 mg, the amount of quercetin is from about 200 mg to about 500 mg, the amount of calcidiol is from about 10 mcg to about 25 mcg, the amount of zinc ascorbate is from about 30 mg to about 190 mg, the amount of myoinositol is from about 200 mg to about 1000 mg and the amount of myricetin is from about 100 mg to about 500 mg wherein the total amount of ingredients/serving (dosage) is the sum of the amounts of individual ingredients in a composition

In another embodiment, a composition is provided. The composition includes hesperidin, quercetin, L-arginine, calcidiol, zinc ascorbate and punicalagin wherein the amount of L-arginine in said compositions is from about 900 mg to about 1000 mg, the amount of hesperidin is from about 100 mg to about 500 mg, the amount of quercetin is from about 200 mg to about 800 mg, the amount of calcidiol is from about 10 mcg to about 25 mcg, the amount of zinc ascorbate is from about 30 mg to about 190 mg, the amount of punicalagin from about 50 mg to about 500 mg wherein the total amount of ingredients/serving (dosage) is the sum of the amounts of individual ingredients in a composition.

Said compositions can be administered to a mammal either in form of a powder, gummy or tablet, in a form of a beverage or syrup or a pharmaceutical formulation. The dietary supplement or the pharmaceutical formulation can be in the form of a tablet, a capsule, a soft chew, or a gel. The composition can also be a food product. Examples include tea (e.g. a tea drink and the contents of a tea bag), soft drinks. juice (e.g., a fruit extract and a juice drink), milk, coffee, jelly, ice cream, yogurt, cookies, cereals, chocolates, and snack bars. Said formulations can also contain various excipients, sweeteners, and artificial flavors.

In still another aspect, an embodiment pertains to a method of administering to a subject in need thereof an effective amount of the embodiment compositions of the present disclosure.

Another embodiment pertains to a composition containing the embodiment compositions of the present disclosure for use in treatment of a medical condition associated with immune system imbalances, and the use of such a composition for the manufacture of a medicament for the just-mentioned treatment.

The details of one or more embodiments of the invention are set forth in the detailed description of the present disclosure below. Other features, objects, and advantages of the invention will be apparent from the description and from the claims.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by embodiments of the present disclosure. As used herein, “about” may be understood by persons of ordinary skill in the art and can vary to some extent depending upon the context in which it is used. If there are uses of the term which are not clear to persons of ordinary skill in the art, given the context in which it is used, “about” may mean up to plus or minus 10% of the particular term.

The terms “%”, “% by weight”, “weight %” and “wt %” are all intended to mean unless otherwise stated, percents by weight based upon a total weight of 100% end composition weight. Thus 10% by weight means that the component constitutes 10 wt. parts out of every 100 wt. parts of total composition.

The terms “treating” and “effective amount”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

All of the embodiments included here are with the proviso that the sum of ingredients in the exemplary compositions does not exceed 100%.

Aspects of the present disclosure include novel combinations, pharmaceutical compositions and methods of for treating and/or preventing viral infections in a mammal where the combinations and pharmaceutical compositions including arginine, vitamin D and antioxidants may increase the production of nitric oxide and decrease oxidative stress by lowering the production of harmful levels of peroxynitrate and increase caveola mediated endocytosis which is a process that upon activation increases the degradation of tumor derived growth factor beta 2 (TGFB2). TGFB2, which, when produced in excess in cells, may exhibit immunosuppressive functions, increase tumor growth and aid the progression of chronic inflammatory disease. Compositions that are capable of increasing caveola mediated endocytosis may have the capacity to decrease TGF beta 2 (TGFB2) levels in a tissue and have therefore therapeutic value by increasing immune system functions and decreasing tumor growth. Quercetin and myricetin may be ABC transport inhibitors. ABC transporter activity may limit the oral bioavailability of provitamin D and that the oral administration of quercetin may result in a significantly higher calcidiol concentration in plasma of patients than the calcidiol concentration observed in patients with the administration of calcidiol without quercetin. The addition of quercetin and/or myricetin in a composition containing calcidiol may increase the oral bioavailability of calcidiol and enhance the therapeutic potential of vitamin D supplementation. Furthermore, arginine mediated may increase in nitric oxide production results in the activation of caveola mediated endocytosis which, in turn, aids the conversion of the vitamin D precursor calcidiol into calcitriol which is the active form of vitamin D which in turn increases endothelial nitric oxide synthase expression and create a positive feedback loop that may increase endothelial nitric oxide production. Thus, the combination of arginine, quercetin myricetin and vitamin D may support a feedback loop that increases endothelial nitric oxide production, vitamin D activation and decreases TGFB2 levels by accelerating its degradation via caveola mediated endocytosis. The TGFB2 lowering activity and immune enhancing activity of embodiments of the present disclosure may be further enhanced by the addition of hesperetin or hesperidin which may inhibit TGFB2 signaling and the expression of this cytokine. Ascorbic acid may slow the metabolism of quercetin and to increase the bioavailability of zinc which has been shown to be important in Treg function and T cell differentiation. Furthermore, quercetin and hesperidin may decrease production of inducible nitric oxide and slow development of inflammation and fibrosis. Accordingly, the combination of the products described herein may have unique cooperative properties that mutually enhance supporting properties of individual constituents and therefore has utility in treatment or prevention of conditions associated with imbalanced immune systems.

Typically, a subject can be administered, once or periodically per day, with one embodiment of the present disclosure in an amount that provides about 900 mg to about 1000 mg, about 900 mg, about 1000 mg of L-arginine, about 200 mg to 500 mg of quercetin; about 100 mg to 500 mg of hesperidin; about 10 mcg to 25 mcg of calcidiol and about 30 mg to 190 mg of zinc ascorbate, wherein the total amount of ingredients/serving (dosage) is the sum of the amounts of individual ingredients in a composition. Examples of subjects include animals, such as, for example, mammals (e.g., humans), birds, reptiles, amphibians, and fish.

Embodiments of the present disclosure can also include 5-hydroxy methyl-2-furfural in an amount that ranges from about 500 mg to about 8000 mg.

Embodiments of the present disclosure can also include punicalagin in an amount that ranges from about 50 mg to about 500 mg, about 100 mg to about 500 mg.

Embodiments of the present disclosure can also include vitamin K in an amount of at least about 70 mcg to about 100 mcg, about 100 mcg.

Embodiments of the present disclosure can also include myoinositol, ferulic acid, myricetin, pterostilbene, resveratrol, capsicum chinense, curcumin, coenzyme Q10, krill oil and herring roe oil each in an amount that ranges from about 100 mg to 800 mg.

Embodiments of the present disclosure can also include about 1 to about 6 billion colony-forming units of each of at least one probiotic selected from the group consisting of Faecalibacterium prausnitzii, Clostridium cluster XIVa, Eubacterium limosum, bifidobacteria, Akkermansia muciniphila.

The term “quercetin” includes both quercetin aglycon and quercetin derivatives such as for example quercetin 3-glucoside. Likewise, the term “hesperidin” includes a flavanone glycoside found in citrus fruits and the term “hesperetin” includes its aglycone. The term “zinc ascorbate” includes the zinc salt of vitamin C (L-ascorbic acid), The term “5-hydroxymethyl-2-furfural” includes hydroxymethylfurfural which is one of the most important products of the maillard reaction. It is preferred that each dose or serving of a first preferred embodiment of the present disclosure contains about 900 mg to about 1000 mg of L-arginine, about 500 mg of quercetin, about 100 mg to about 500 mg of hesperidin or hesperetin, about 10 mcg to about 25 mcg (micrograms) calcidiol, about 30 mg to about 190 mg zinc ascorbate.

It is preferred that each dose or serving of a second preferred embodiment of the present disclosure contains about 900 mg to about 1000 mg of L-arginine, about 500 mg of quercetin, about 100 mg to about 500 mg of hesperidin or hesperetin, about 10 mcg to about 25 mcg (micrograms) calcidiol, about 30 mg to about 190 mg zinc ascorbate, and about 1000 mg to 8000 mg of 5-hydroxymethyl-2-furfural

It is preferred that each dose or serving of a third preferred embodiment of the present disclosure contains about 900 mg to about 1000 mg of L-arginine, about 500 mg of quercetin, about 100 mg to about 500 mg of hesperidin or hesperetin, about 10 mcg to about 25 mcg (micrograms) calcidiol, about 30 mg to about 190 mg zinc ascorbate, and about 100 mg to 500 mg of punicalagin

A pharmaceutical composition and embodiments, including method embodimenmts, thereof of the present disclosure can be directly using a variety of drug formulations. Such drug formulation contains combinations of ingredients of the embodiments of the present disclosure. Further, the drug formulation can be produced by an arbitrary method that has been well known in the art of drug formulation by mixing the active ingredients of the embodiments of the present disclosure with at least one type of pharmacologically acceptable carrier, vehicle or bioavailability enhancing cyclodextrine. It is desirable that the most effective administration route of drug formulation would be selected for treatment. Examples thereof include oral administration, topical administration and parenteral administration such as intravenous, intraperitoneal, or subcutaneous administration. However, oral administration for treatment of medical conditions in and out of hospital settings is preferable. Examples of dosage forms that can be used for administration include: oral agents such as tablets, powders, granules, pills, suspensions, emulsions, infusions and decoctions, capsules, gummies, syrups, liquid, elixirs, extracts, tinctures, and fluid extracts; and parenteral agents such as parenteral injections, intravenous fluids, creams, and suppositories. The compositions of the embodiments of the present disclosure in the form of an oral composition is preferably used.

In the case involving the use of liquid preparations such as syrup appropriate for oral administration, the preparations can be formulated by addition of water, Sugars such as sucrose, sorbitol, and fructose; glycols such as polyethylene glycol and propylene glycol, oils such as sesame oil, olive oil, and soybean oil; antiseptics such as p-hydroxy benzoate esters; parahydroxy benzoate derivatives such as methyl parahydroxy benzoate; preservatives such as sodium benzoate; and fruit flavors.

In addition, in the case involving the use of tablets, powders, and granules that are appropriate for oral administration the preparations can be formulated by addition of sugar such as lactose, glucose, sucrose, mannitol, and sorbitol; starch from potatoes, wheat, and corn; an inorganic substance such as calcium carbonate, calcium sulfate, sodium bicarbonate, and sodium chloride; an excipient of a plant-derived powder such as crystalline cellulose, a sweetroot powder and gentian powder, a disintegrator such as starch, agar, gelatin powder, crystalline cellulose, carmellose sodium, carmellose calcium, calcium carbonate, sodium bicarbonate, and sodium alginate; a lubricant such as magnesium stearate, talc, hydrogenated plant oil, macrogol, and silicone oil; a binder such as polyvinyl alcohol, hydroxypropyl cellulose, methyl cellulose, ethylcellulose, carmellose, gelatin, and starch paste liquid; a surfactant such as fatty acid ester, and a plasticizer such as glycerine.

In addition, in the case involving the use of tablets, powders, and granules that are appropriate for oral administration the preparations can be formulated by addition of health promoting bacteria belonging to the genus, Faecalibacterium prausnitzii, Clostridium cluster XIVa also known as Clostridium coccoides group, Eubacterium limosum, bifidobacteria, Akkermansia muciniphila.

It is also possible to add an additive generally used for foods and beverages to the embodiments of the present disclosure appropriate for oral administration. Examples of additives include sweeteners, colorants, preservatives, thickening stabilizers, antioxidants, coloring agents, bleaches, antifungal agents, gumbases, bittering agents, enzymes, gloss agents, acidulants, seasonings, emulsifiers, fortifiers, production agents, aroma chemicals, and spice extracts.

The embodiments of the present disclosure appropriate for oral administration may be directly used in the form of, for example, a powder food product, a sheet-type food product, a bottled food product, a canned food product, a retort food product, a capsule food product, a tablet food product, a liquid food product, or a drink. In addition, the drug formulation may be used in the form of food or beverage such as health food, functional food, nutritional supplement, or food for specified health use. For example, a parenteral injection appropriate for parenteral administration comprises preferably a sterilized aqueous agent which contains an embodiment of the present disclosure and which is isotonic to the blood of a recipient. For example, for a parenteral injection, an injectable solution is prepared with the use of a pharmaceutically acceptable carrier or vehicle comprising a salt solution, a glucose solution, or a mixture of a salt solution and a glucose solution.

In addition, it is also possible to add at least one supplemental component to a parenteral agent, wherein such components can be selected from the group consisting of diluents, antiseptics, flavors, excipients, disintegrators, lubricants, binders, surfactants, and plasticizers, which are described above for an oral agent.

The embodiments of the present disclousre can be in various other forms. For example, it can be a soft chew composition that includes sugar, corn syrup, sucralose, soy lecithin, corn starch, glycerin, palm oil, xylitol, carrageenan, FD&C Yellow #6, FD&C Yellow #5, and natural and/or artificial flavors, and contain additional natural products supporting the immune systems the such as for example icariin, myoinositol, ferulic acid, myricetin, pterostilbene, resveratrol, capsicum chinense, curcumin, coenzyme Q10, eicosapentaenoic acid, vanillic acid, docosahexaenoic acid, linoleic acid, krill oil and herring roe oil. For example, the amount of icariin can be from about 10 mg to about 100 mg, about 60 mg.

An “effective amount” also refers to a dose of the composition that is sufficient to provide a physical benefit (e.g., lowering C-reactive protein levels; lowering TNF alfa levels or increasing interleukin 10 levels). Both in vivo and in vitro studies can be conducted to determine optimal administration routes and doses.

Embodiments of the present disclosure include a method and a pharmaceutical composition used therefor for supporting physical or mental health in a subject by reducing circulating levels of C-reactive protein, pro-inflammatory cytokine TNF alpha, and increasing circulating levels of anti-inflammatory cytokine interleukin 10 (IL-10) including administering to the subject in need of such physical or mental health enhancing effective amount of a composition included in the present disclosure. The physical or mental health conditions or maladies that can be enhanced include, for example, aging, alopecia, rheumatoid arthritis, psoriasis, atherosclerosis, type 1 and type 2 diabetes, chronic fatigue, Irritable bowel syndrome, colitis, nephropathies, dementias, immune system disorders, long covid and hepatic dysfunctions.

Embodiments of the present disclosure include a method and a pharmaceutical composition used therefor for treatment for lowering levels of C-reactive protein, lowering levels of TNF alpha and increasing levels of IL10 including administering to a subject in need of such treatment an effective C-reactive protein and/or TNF alpha lowering amount of a composition included in the present disclosure including administering to a subject in need of such treatment an effective amount of an embodiment of the present disclosure. The conditions or maladies that can be affected by lowering levels of C-reactive protein and/or TNF alpha include, for example, an autoimmune disease such as systemic lupus erythematosus, rheumatic arthritis, systemic sclerosis, type 1 diabetes. infections or infectious diseases such as for example, leprosy, Whipple's disease, influenza, severe acute respiratory syndrome, malaria, human metapneumovirus, picornavirus, Mycoplasma pneumoniae, Chlamydia, parainfluenza virus, respiratory syncytial virus, adenovirus, zika virus, dengue virus, inflammatory diseases such as for example atopic dermatitis, inflammation, asthma, pulmonary fibrosis, prostatitis, inflammatory bowel disease, stress, aging, alopecia, arthritis including rheumatoid arthritis, psoriasis, dermatitis, atherosclerosis, type 1 and type 2 diabetes, chronic fatigue syndrome, Irritable bowel syndrome, colitis, nephropathies, dementias, immune system disorders, long covid, sexual dysfunctions, chronic constipation, periodontal disease, cerebrovascular disease, atherosclerosis, Parkinson's disease, Alzheimer's disease, dementias and hepatic dysfunctions

Embodiments of the present disclosure include a method and a pharmaceutical composition used therefor for treating or preventing diseases or disorders selected from the group consisting of psoriasis, atopic dermatitis, asthma, emphysema, pulmonary fibrosis, rheumatoid arthritis, atherosclerosis, diabetes, infections, sepsis, prostatitis, periodontitis, long covid, Crohn disease, ulcerative colitis and depression, sleep including administering to a subject in need of such treatment an effective amount of an embodiment of the present disclosure.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

All publications, including but not limited to, issued patents, patent applications, and journal articles, cited in this application are each herein incorporated by reference in their entirety.

Thus, while there have been shown, described and pointed out, fundamental novel features of the present disclosure as applied to the exemplary embodiments thereof, it will be understood that various omissions and substitutions and changes in the form and details of devices and methods illustrated, and in their operation, may be made by those skilled in the art without departing from the spirit or scope of the present disclosure. Moreover, it is expressly intended that all combinations of those elements and/or method steps, which perform substantially the same function in substantially the same way to achieve the same results, are within the scope of the present disclosure. Moreover, it should be recognized that structures and/or elements and/or method steps shown and/or described in connection with any disclosed form or embodiment of the present disclosure may be incorporated in any other disclosed or described or suggested form or embodiment as a general matter of design choice. It is the intention, therefore, to be limited only as indicated by the scope of the claims appended hereto.

This written description uses examples as part of the disclosure, including the best mode, and to enable any person skilled in the art to practice the disclosed implementations, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A composition comprising quercetin, hesperidin, L-arginine, calcidiol, and zinc ascorbate wherein the amount of L-arginine is from about 900 mg to about 1000 mg, the amount of hesperidin is from about 100 mg to about 500 mg, the amount of quercetin is from about 300 mg to about 800 mg, the amount of calcidiol is from about 10 mcg to about 25 mcg, and the amount of zinc ascorbate is from about 30 mg to about 190 mg.

2. The composition according to claim 1, wherein the amount of L-arginine is about 1000 mg, the amount of hesperidin is about 300 mg, the amount of quercetin is about 500 mg, the amount of calcidiol is about 25 mcg, and the amount of zinc ascorbate is about 95 mg.

3. The composition according to claim 1, wherein the amount of L-arginine is about 900 mg, the amount of hesperidin is about 300 mg, the amount of quercetin is about 500 mg, the amount of calcidiol is about 25 mcg, and the amount of zinc ascorbate is about 95 mg.

4. The composition according to claim 1, further including icariin wherein the amount of L-arginine is about 1000 mg, the amount of hesperidin is about 200 mg, the amount of quercetin is about 500 mg, the amount of calcidiol is about 25 mcg, the amount of zinc ascorbate is about 95 mg, and the amount of icariin is about 60 mg.

5. The composition according to claim 1, further including 5-hydroxy methyl furfural wherein the amount of L-arginine is about 1000 mg, the amount of hesperidin is about 300 mg, the amount of quercetin is about 500 mg, the amount of 5-hydroxy methyl furfural is from about 500 mg to about 5 g, the amount of calcidiol is about 12.5 mcg, and the amount of zinc ascorbate is about 190 mg.

6. The composition according to claim 1, further including punicalagin wherein the amount of L-arginine is about 1000 mg, the amount of hesperidin is about 300 mg, the amount of quercetin is about 500 mg, the amount of punicalagin is from about 100 mg to about 500 mg, the amount of calcidiol is about 12.5 mcg, and the amount of zinc ascorbate is about 190 mg.

7. The composition according to claim 1, further including about 100 mcg of vitamin K.

8. The composition according to claim 2, further containing about 100 mcg of vitamin K.

9. The composition according to claim 3, further including at about 100 mcg of vitamin K.

10. The composition according to claim 4, further including about 100 mcg of vitamin K.

11. The composition according to claim 5, further including about 100 mcg of vitamin K.

12. A composition according to claim 1, further including about 100 mg to about 800 mg of each of at least one ingredient selected from the group consisting of myoinositol, ferulic acid, myricetin, pterostilbene, resveratrol, capsicum chinense, punicalagin, curcumin, coenzyme Q10, vanillic acid, krill oil, herring roe oil, krill oil containing krill protein, herring roe protein, mung bean protein, ginkgo biloba.

13. A composition according to claim 12, further including about 1 to about 6 billion colony-forming units of each of at least one probiotic selected from the group consisting of Faecalibacterium prausnitzii, Clostridium cluster XIVa, Eubacterium limosum, bifidobacteria, Akkermansia muciniphila.

14. The composition according to claim 1, wherein the composition is in a powder form.

15. The composition according to claim 1, wherein the composition is in liquid form.

16. The composition according to claim 15 wherein the liquid form is tea, soft drinks, juice, milk, coffee, jelly, ice cream, yogurt, cookies, cereals, chocolates, snack bars or gummy.

17. A method for treating alopecia, rheumatoid arthritis, psoriasis, atopic dermatitis, atherosclerosis, type 1 and type 2 diabetes, chronic fatigue syndrome, Irritable bowel syndrome, colitis, nephropathies, dementias, immune system disorders, long covid and, hepatic dysfunctions and aging associated morbidity, comprising administering to a subject in need of such treatment an effective amount of the composition according to claim 1.

18. A method of treatment for lowering C-reactive protein levels, comprising administering to a subject in need of such treatment a C-reactive protein level lowering effective amount of the composition according to claim 1.

19. A method of treatment for lowering TNF alpha levels, comprising administering to a subject in need of such treatment a TNF alpha level lowering effective amount of the composition according to claim 1.

20. A method of treatment for increasing interleukin 10 levels, comprising administering to a subject in need of such treatment an interleukin 10 increasing effective amount of the composition according to claim 1.

21. A method of treating a disease or disorder selected from the group consisting of an autoimmune disease, atopic dermatitis, alopecia, proriasis, asthma, pulmonary fibrosis, prostatitis, rheumatoid arthritis, inflammatory bowel disease, arthritis, type 1 and type 2 diabetes, sexual dysfunctions, chronic constipation, infectious diseases, periodontal disease, cerebrovascular disease, atherosclerosis, Parkinson's disease, Alzheimer's disease and dementias, comprising of administering to a subject in need of such treatment an effective amount of a composition according to claim 1.

22. The composition according to claims 1, wherein a dosage is the sum of the amounts of ingredients in the composition.
